(19) United States
(12) Patent Application Publication
(10) Pub. No.: US 2019/0282207 A1
(43) Pub. Date: Sep. 19, 2019

(54) HIGH INTENSITY FOCUSED ULTRASOUND (HIFU) DEVICE AND SYSTEM

(71) Applicant: Butterfly Network, Inc., Guilford, CT (US)

(72) Inventors: Kailiang Chen, Branford, CT (US); Nevada J. Sanchez, Guilford, CT (US); Christopher Thomas McNulty, Guilford, CT (US)

(73) Assignee: Butterfly Network, Inc., Guilford, CT (US)

(21) Appl. No.: 16/432,901

(22) Filed: Jun. 5, 2019

Related U.S. Application Data
(63) Continuation of application No. PCT/US2017/064862, filed on Jun. 12, 2017.
(60) Provisional application No. 62/431,379, filed on Dec. 7, 2016.

(51) Int. Cl. A61B 8/00; A61N 7/02

(52) U.S. Cl. CPC A61B 8/4483; A61B 8/4272; A61B 8/4227; A61N 7/02

(57) ABSTRACT

An exemplary system includes a first ultrasonic transducer assembly configured to deliver high intensity focused ultrasonic (HIFU) energy to a point of interest within a subject, and a second ultrasonic transducer assembly configured to perform imaging of the subject. In another embodiment, a housing is configured to receive an ultrasound probe. The housing may include a cooling circuit and power supply for the ultrasound probe.

### CROSS-REFERENCE TO RELATED APPLICATIONS

This application is a Continuation of Patent Application Serial No. PCT/US2017/064862, filed Dec. 6, 2017 under Attorney docket No. B1348.70038WO00, and entitled “HIGH INTENSITY FOCUSED ULTRASOUND (HIFU) DEVICE AND SYSTEM,” which is hereby incorporated herein by reference in its entirety.

PCT/US2017/064862 claims the benefit of U.S. Provisional Patent Application Ser. No. 62/431,379, filed Dec. 7, 2016 under Attorney Docket No. B1348.70038US00, and entitled “HIGH INTENSITY FOCUSED ULTRASOUND (HIFU) DEVICE AND SYSTEM,” which is hereby incorporated herein by reference in their entirety.

### FIELD

The present disclosure relates generally to ultrasound technology. In particular, the present disclosure relates to a high intensity focused ultrasound (HIFU) device and system.

### BACKGROUND

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using a probe), sound waves are reflected off the tissue with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

With respect to treatment, as an alternative to more invasive types of surgical procedures, many physicians are employing the use of high intensity focused ultrasound (HIFU) as a technique to therapeutically treat internal body tissues. With HIFU, an ultrasound signal of sufficient power (e.g., pressure and velocity) and time is focused on a target volume of tissue in order to change a state of the tissue by rapid heating and/or mechanical destruction by cavitation. The treated tissue may form one or more lesions that may be left in the body and thereafter absorbed through normal physiological processes.

In order to effectively treat tissue, the energy of the delivered HIFU signal must be sufficient to cause the desired physical effect(s). On the other hand, the delivered energy should not be too large or uncontrolled so as to cause unintended collateral damage to healthy tissues surrounding the target volume. The non-homogenous nature of tissue(s) in the body creates variations in attenuation, propagation velocity, and acoustic impedance that modify the expected acoustic wave propagation and deposition of HIFU energy delivered to a target tissue volume when compared to homogeneous material. Thus, certain treatment regimens that are solely based on applying a predetermined dose of HIFU energy may therefore achieve inconsistent results due to such variations.

### SUMMARY

In one embodiment, a system includes a first ultrasonic transducer assembly configured to deliver high intensity focused ultrasonic (HIFU) energy to a point of interest within a subject, and a second ultrasonic transducer assembly configured to perform imaging of the subject.

In another embodiment, a system includes a plurality of high intensity focused ultrasonic (HIFU) units, each configured to deliver high energy focused ultrasound energy to a point of focus; and receive circuitry configured to determine a relative alignment between individual HIFU units so as to implement a self-calibration with respect to a transmit phase of the individual HIFU units.

### BRIEF DESCRIPTION OF THE DRAWINGS

Various aspects and embodiments of the disclosed technology will be described with reference to the following Figures. It should be appreciated that the figures are not necessarily drawn to scale. Items appearing in multiple figures are indicated by the same reference number in all the figures in which they appear.

FIG. 1 is a perspective view of a handheld ultrasound probe suitable for use with exemplary embodiments;

FIG. 2 is an exploded perspective view of the ultrasound probe of FIG. 1;

FIG. 3 is a partial cross-sectional view of an exemplary ultrasound-on-chip device suitable for use with exemplary embodiments;

FIG. 4 is a perspective view of another type of ultrasound probe suitable for use with exemplary embodiments;

FIG. 5 illustrates the ultrasound probe of FIG. 4 affixed to a patient;

FIG. 6 is a top view illustrating an alternative fastening mechanism for the ultrasound probe of FIG. 4;

FIG. 7 illustrates the ultrasound probe of FIG. 4 affixed to the patient;

FIG. 8 is an exploded perspective view of the ultrasound probe of FIG. 6;

FIGS. 9-11 are perspective views of a HIFU apparatus in accordance with exemplary embodiments;

FIG. 12 is a schematic diagram illustrating an exemplary application of a HIFU apparatus, in accordance with embodiments; and

FIGS. 13-18 illustrate various power and acoustic pressure field simulations using different numbers of HIFU transducer chips.

### DETAILED DESCRIPTION

Disclosed herein are embodiments of a fully integrated HIFU Array, device and system. Among several advantages of the disclosed embodiments are, for example: the observation and tracking of targets from ultrasonic imaging; the use of an electronic array with flexible focusing of targets; and a multi-chip assembly that may be adapted to different clinical applications. For example, several HIFU chips may be tiled into a large aperture to produce higher delivered energy and better focusing. In one specific example, a curved aperture may be configured for use in brain related therapy, in which phase adjustment techniques may be applied to address any chip-to-chip misalignment.

Still another advantage that may be gained with respect to conventional ultrasonic transducers formed from piezoelectric materials is the use of capacitive micromachined ultrasonic transducers (CMUTs) formed from a semiconductor substrate. In the case of a CMUT device, a flexible membrane is suspended above a conductive electrode by a small gap. When a voltage is applied between the membrane and the electrode, Coulombic forces attract the flexible membrane to the electrode. As the applied voltage varies over time, so does the membrane position, thereby generating acoustic energy that radiates from the face of the transducer as the membrane moves. More specifically, one advantage arising from the use of CMUTs is a smaller degree of self-heating, as silicon has less of impedance mismatch to a medium, and better thermal conductivity with respect, to PZT. Furthermore, capacitive sensors have lower electrical losses/heating with respect to piezo counterparts.

From a manufacturing standpoint, CMUTs have the further benefit of low-cost, scalable semiconductor fabrication, as well as the ability to implement 2D arrays and flexible interconnection(s) with electronics. In contrast, with PZT technology, manual dicing is required, and it is difficult to interconnect for 2D arrays. In addition, PZT devices have a relatively large kerf between elements and less active area.

Embodiments of the present disclosure are described more fully hereinafter with reference to the accompanying drawings, in which some, but not all, embodiments of the present disclosure are shown. Indeed, the present disclosure can be embodied in many different forms and should not be construed as limited to the embodiments set forth herein. Rather, these embodiments are provided so that this disclosure clearly satisfies applicable legal requirements. Like numbers refer to like elements throughout.

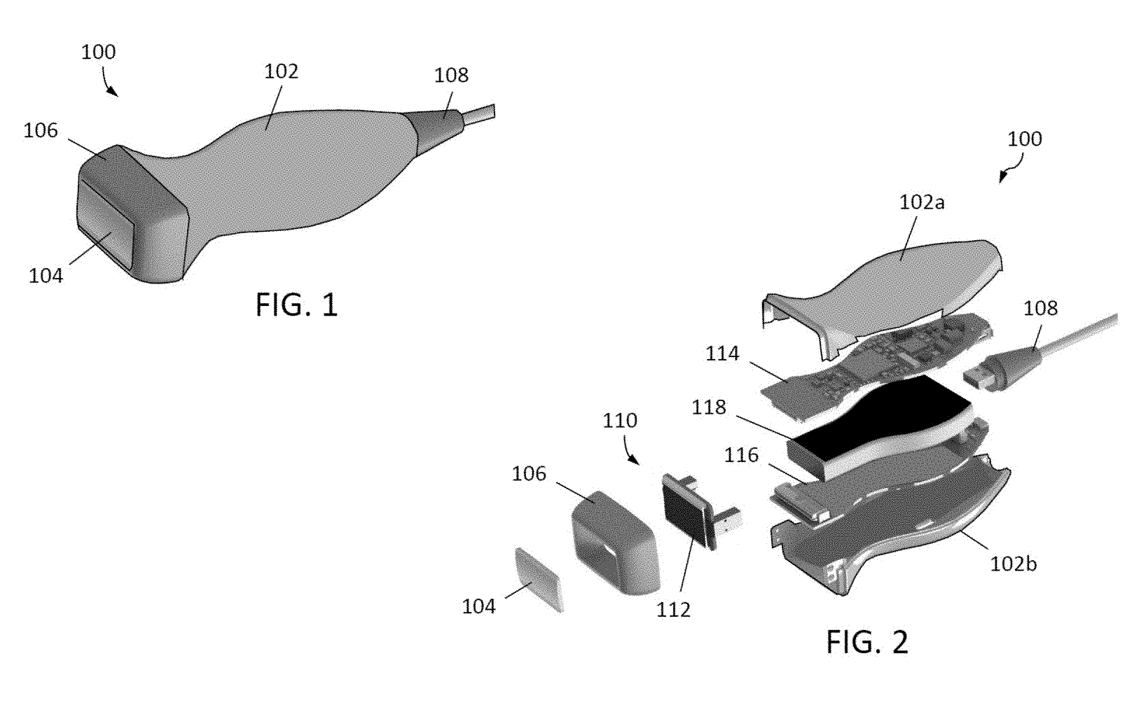

Referring initially to FIG. 1 and FIG. 2, an exemplary ultrasound probe 100 is depicted in a perspective view and an exploded perspective view, respectively. It should be understood, however, that the ultrasound probe 100 depicted in FIG. 1 and FIG. 2 represents one exemplary application for the acoustic attenuation features described herein, and other form factors, applications and devices are also contemplated. As shown in FIG. 1, the exemplary ultrasound probe 100 is a handheld probe that includes a probe housing 102 having an acoustic lens 104 and shroud 106 disposed at a first end thereof, and a cable assembly 108 disposed at a second end thereof. The shroud 106 prevents direct contact between an ultrasonic transducer assembly 110 (FIG. 2) and a patient (not shown) when the ultrasound probe 100 is used to image and/or provide therapy to the patient.

In addition to imaging, the acoustic lens 104 may also be configured to focus acoustic energy to spots having areas of the size required for high-intensity focused ultrasound (HIFU) procedures. Furthermore, the acoustic lens 104 may acoustically couple the ultrasonic transducer assembly 110 to the patient (not shown) to minimize acoustic reflections and attenuation. In some embodiments, the acoustic lens 104 may be fabricated with materials providing impedance matching between ultrasonic transducer assembly 110 and the patient. In still other embodiments, the acoustic lens 104 may provide electric insulation and may include shielding to prevent electromagnetic interference (EMI). Additionally, the shroud 106 and acoustic lens 104 may provide a protective interface to absorb or reject stress between the ultrasonic transducer assembly 110 and the acoustic lens 104.

As also shown in FIG. 2, the ultrasonic transducer assembly 110 includes an ultrasound-on-chip device 112 having an ultrasonic transducer array that is covered by the acoustic lens 104 when the ultrasound probe 100 is assembled. An interior region of the ultrasound probe 100, encapsulated by upper probe housing section 102a and lower probe housing section 102b, may also include components such as a first circuit board 114, a second circuit board 116 and a battery 118. The circuit boards 114 and 116 may include circuitry configured to operate the ultrasonic transducer arrangement 110 in a transmit mode to transmit ultrasound signals, or receive mode, to convert received ultrasound signals into electrical signals. Additionally, such circuitry may provide power to the ultrasonic transducer assembly 110, generate drive signals for the ultrasonic transducer assembly 110, process electrical signals produced by the ultrasonic transducer assembly 110, or perform any combination of such functions. The cable assembly 108 may carry any suitable analog and/or digital signals to and from circuit boards 114 and 116.

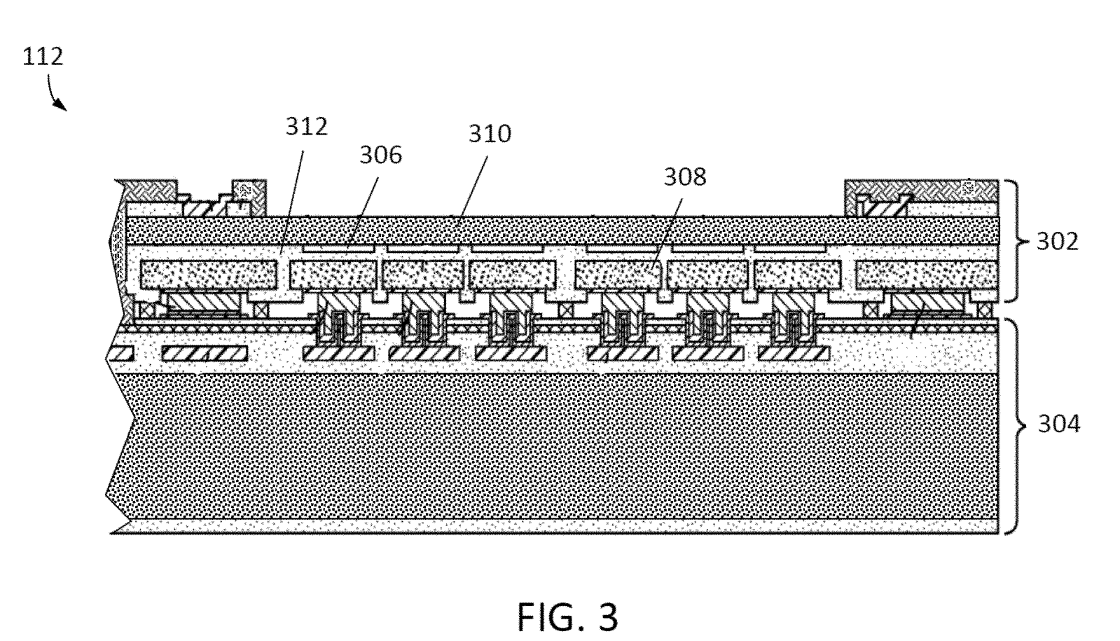

One possible configuration for the ultrasound-on-chip device 112 is illustrated in the partial cross-sectional view of FIG. 3. In the embodiment depicted, the ultrasound-on-chip device 112 includes an ultrasonic transducer substrate 302 that is bonded to an integrated circuit substrate 304, such as a complementary metal oxide semiconductor (CMOS) substrate for example. The ultrasonic transducer substrate 302 has a plurality of cavities 306 formed therein, and is an example of a CMUT device as described above. The cavities 306 are formed between a first silicon device layer 308 and a second silicon device layer 310. A silicon oxide layer 312 (e.g., a thermal silicon oxide such as a silicon oxide formed by thermal oxidation of silicon) may be formed between the first and second silicon device layers 308 and 310, with the cavities 306 being formed therein. In this non-limiting example, the first silicon device layer 308 may be configured as a bottom electrode and the second silicon device layer 310 may be configured as a membrane. Thus, the combination of the first silicon device layer 308, second silicon device layer 310, and cavities 306 may form an ultrasonic transducer (e.g., a CMUT), of which six are illustrated in this non-limiting cross-sectional view. To facilitate operation as a bottom electrode or membrane, one or both of the first silicon device layer 308 and second silicon device layer 310 may be doped to act as conductors, and in some cases are highly doped (e.g., having a doping concentration greater than 10¹⁵ dopants/cm³ or greater).

In terms of the aforementioned forward direction toward a subject being imaged and/or having therapy applied thereto, this would be in the upward direction with respect to the view in FIG. 3, whereas the backward direction away from the subject being imaged and/or having therapy applied thereto would be in the downward direction with respect to the view in FIG. 3. Additional information regarding the fabrication and integration of CMUTs with CMOS wafers may be found in U.S. Pat. No. 9,067,779, assigned to the assignee of the present application, the contents of which are incorporated by reference herein in their entirety. Again however, it should be appreciated that the ultrasonic transducer substrate 302/CMOS substrate 304 embodiment represents just one possible configuration for the ultrasound-on-chip device 112. Other configurations are also possible including, but not limited to, a side-by-side arrangement where transducers and CMOS circuitry are formed on a same substrate, as well as arrays formed from piezoelectric micromachined ultrasonic transducers (PMUTs), or other suitable types of ultrasonic transducers. In still other embodiments, the ultrasound-on-chip device 112 may include an ultrasonic transducer array by itself (i.e., an ultrasonic transducer chip), where CMOS circuitry is located on a different substrate or circuit board altogether.

Other examples of suitable ultrasound on a chip systems for use in HIFU are described in U.S. Pat. No. 9,521,991, which is assigned to the Assignee of the current application and which is incorporated herein by reference in its entirety. U.S. Pat. No. 8,852,103 also describes HIFU, is assigned to the Assignee of the present application, and is incorporated herein by reference in its entirety.

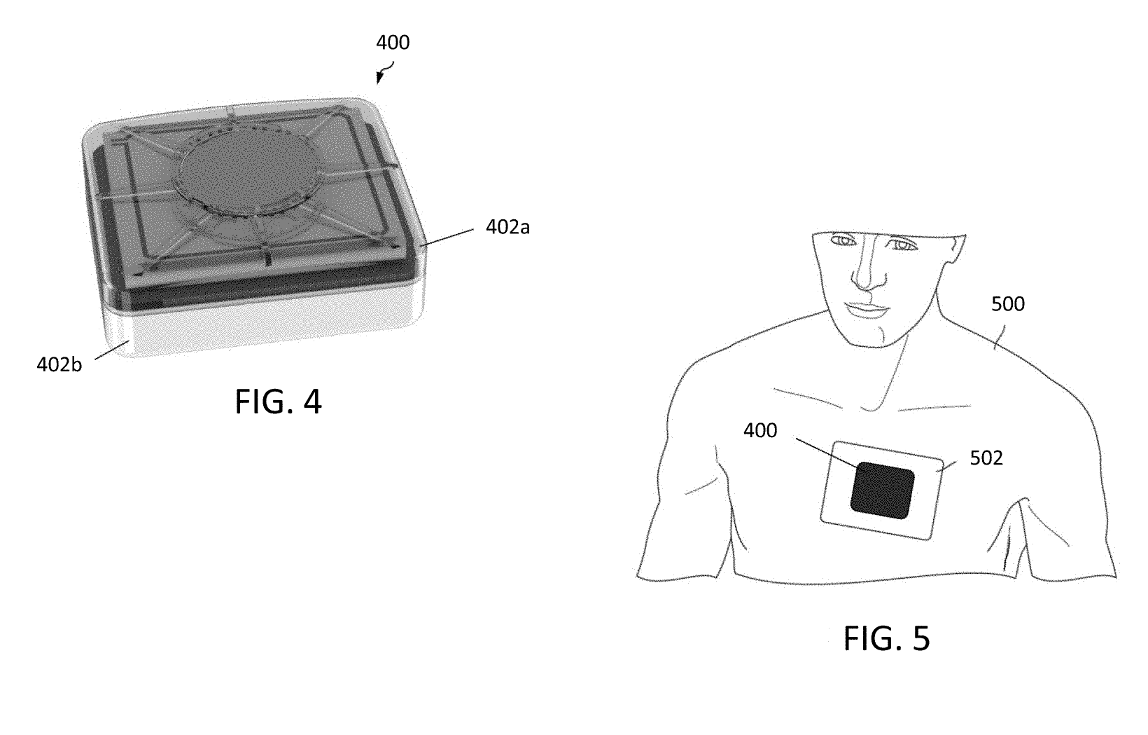

In addition to handheld probe embodiments such as depicted in FIG. 1 and FIG. 2, it is further contemplated that other ultrasound probe form factors may incorporate HIFU functionality as described hereinafter. For example, FIG. 4 is a perspective view illustrating an ultrasound probe 400 that is embodied in a patch configuration, and having an upper housing 402a and a lower housing 402b. The probe 400 is shown coupled to a patient 500 in FIG. 5, and may be configured to wirelessly communicate with one or more external devices. In the example depicted, the probe 400 may also be provided with dressing 502 that provides an adhesive surface for both the probe housing as well as to the skin of the patient. One non-limiting example of such a dressing 502 is Tegaderm™, a transparent medical dressing available from 3M Corporation. Although not specifically shown in FIG. 5, the lower housing 402b may include an opening that aligns with a corresponding opening in the dressing 502 so that transducer elements of the ultrasound probe 400 may be acoustically coupled to the patient 500.

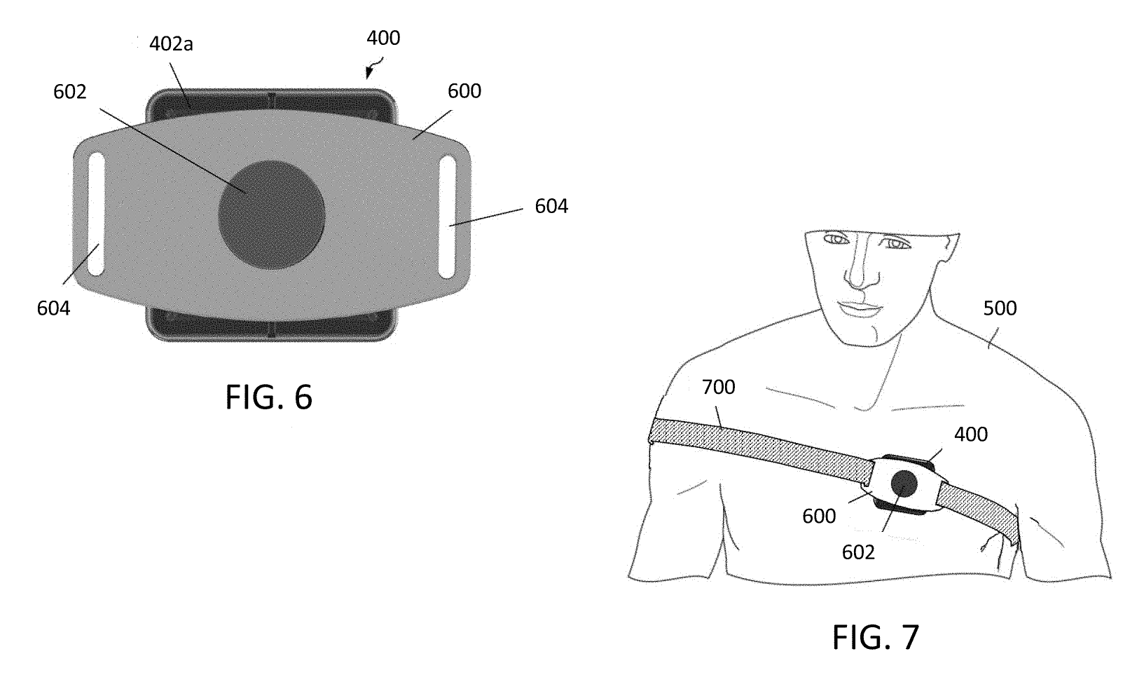

Referring to FIG. 6, an alternative fastening mechanism for the ultrasound probe 400 is illustrated. In the embodiment shown, the ultrasound probe 400 further includes a buckle 600 affixed to the upper housing 402a via a post 602 using, for example, a threaded engagement between the buckle 600 and the post 602. Other attachment configurations are also contemplated, however. As further shown in FIG. 16, the buckle 600 includes a pair of slots 604 that in turn accommodate a strap 700 (FIG. 7). In this example, the strap 700 is wrapped around the patient 500 and appropriately tightened in order to secure the ultrasound probe 400 to a desired location on the patient 1500 for acquisition of desired ultrasound data and/or delivery of desired ultrasound energy.

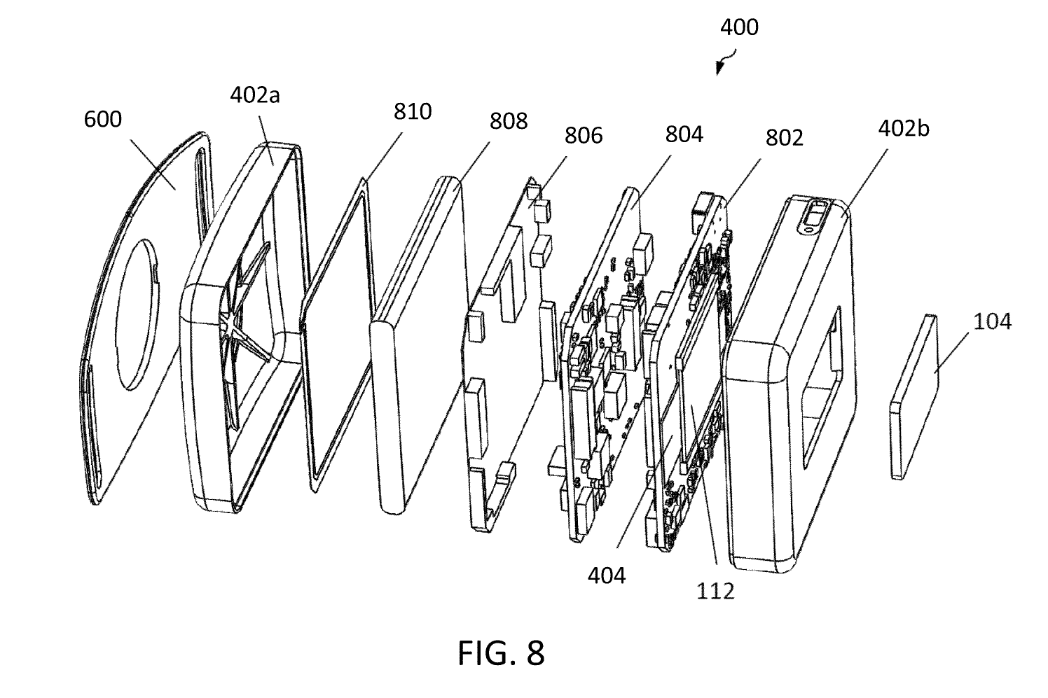

FIG. 8 illustrates an exploded perspective view of the ultrasound probe 400 of FIG. 6. For ease of illustration and comparison, similar components with respect to the embodiment of FIG. 1 and FIG. 2 are designated with similar reference numerals. For example, in addition to the upper housing 402a, lower housing 402b and buckle, the ultrasound probe 400 further includes an acoustic lens 104 to cover the ultrasound-on-chip device 112, which in turn is attached to a heat sink device 404. In contrast to the handheld probe embodiment of FIGS. 1-2 in which the ultrasonic transducer assembly 110 includes an interposer circuit board 402, the ultrasound-on-chip device 112 and heat sink device 404 are attached directly to a first circuit board 1802. In addition, the ultrasound probe 400 further includes, by way of example, a second circuit board 804 (e.g., for power supply components), an insulator board 806, battery 808 and antenna (e.g., to enable wireless communication to and from the ultrasound probe 400).

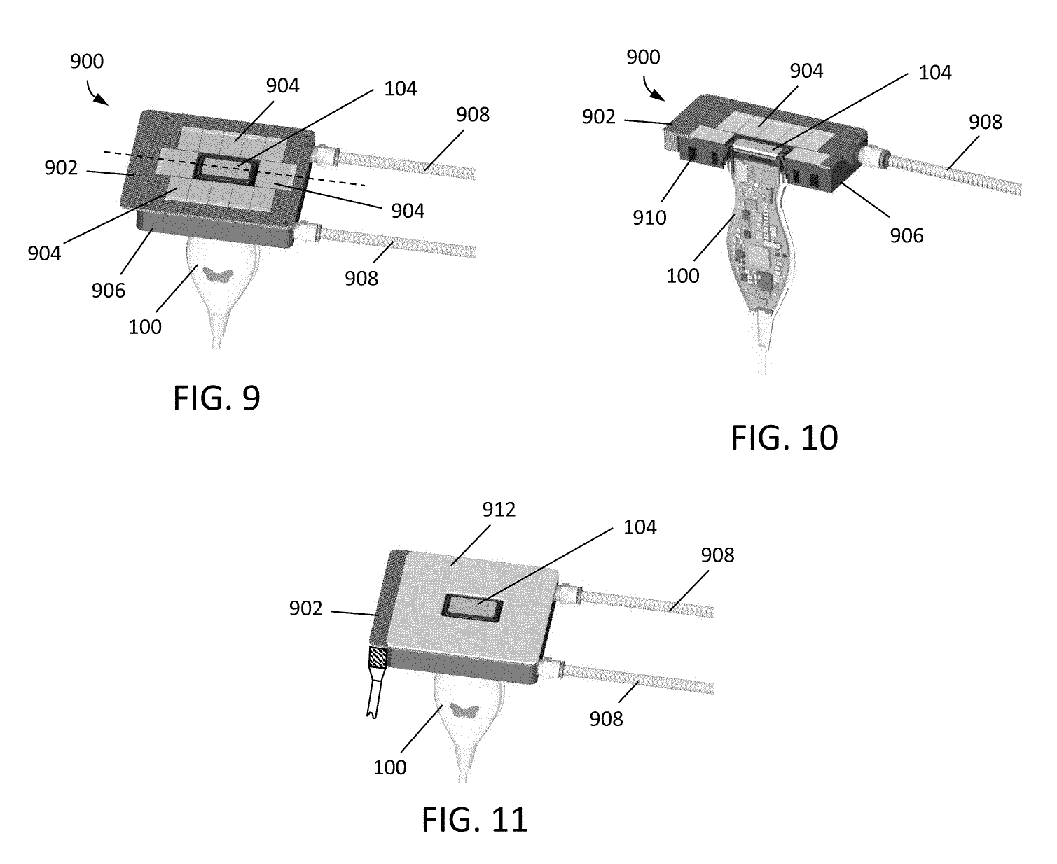

Referring now to FIG. 9 and FIG. 10, there is shown a perspective view of a HIFU apparatus 900 in accordance with exemplary embodiments. In the embodiment depicted, the apparatus 900 includes a circuit board 902 having one or more HIFU transducer chips 904 mounted thereon. The HIFU transducer chips 904 are also in thermal contact with a cooling block 906 (e.g., copper or other thermally conductive material(s)). Cooling lines 908 may be used to circulate a coolant material (not shown) through one or more channels 910 of the cooling block 906, as best seen in FIG. 10. The number of HIFU transducer chips provided with the apparatus 900 is not necessarily limited to any specific number as depicted in the illustrated embodiments, and may be selected based on the desired application(s). As further illustrated in FIG. 9 and FIG. 10, an ultrasound probe (e.g., probe 100) may be inserted through an opening in the cooling block 906 and circuit board 902 such that the acoustic lens 104 of the probe 100 is substantially co-planar with the plurality of HIFU transducer chips 904. In this manner, the HIFU apparatus 900 may provide both imaging functionality (via the probe 100) and HIFU therapy functionality (via the HIFU transducer chips 904). In other embodiments, the HIFU transducer chips 904 need not necessarily be co-planar with one another or co-planar with the acoustic lens 104 of the probe 100. As shown in FIG. 11, an acoustic lens 912 or other capping material may be formed over the HIFU transducer chips 904, optionally with an opening therein to allow the acoustic lens 104 of the probe 100 to protrude therethrough. FIG. 11 also illustrates an connector 914 and cable 916 that may attach to the circuit board 902 to deliver power and signals to/from the HIFU transducer chips 904.

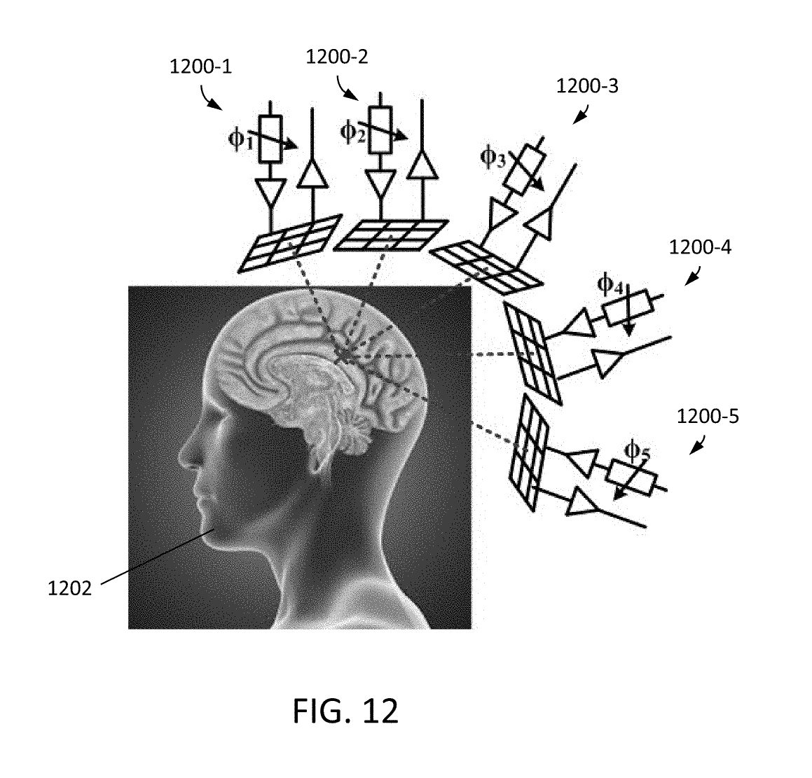

FIG. 12 is a schematic diagram illustrating an exemplary application of a HIFU apparatus, in accordance with embodiments. The example depicted is illustrative of various capabilities, including (but not limited to): the ability to flexibly focus with a 2D array probe to provide treatment monitoring; the use of multiple HIFU chips collaborating to deliver acoustic energy at a common focus; and multi-chip adaptive phase adjustment. As shown in FIG. 12, a plurality of HIFU units 1200-1, 1200-2, 1200-3, 1200-4, 1200-5 is configured to direct HIFU energy at a single point of focus within a patient 1202. In the example illustrated, the point 1204 of focus is within the brain of the patient 1202. It should be appreciated that each HIFU unit may have one or more individual HIFU transducer chips associated therewith, and that the specific number of HIFU units depicted in FIG. 12 is exemplary only.

It may further be appreciated that in the application of HIFU energy to the brain of the patient, the apparatus may be configured such that individual HIFU chips or HIFU units may not be arranged in a planar manner, but instead arranged in a manner to conform to an anatomical structure of the patient 1202 (e.g., a rounded anatomical structure such as a patient's head). Here, the relative location of chips affect the relative phase relationship between the chips/units. Accordingly, embodiments herein provide the capability the self-calibration of phase (e.g., between transmit circuits ϕ₁, ϕ₂, ϕ₃, ϕ₄, ϕ₅). Regardless of whether the HIFU transducer chips or units are co-planar, it is contemplated that any misalignment of the chips/units may utilize an adaptive, auto-correcting phase adjustment. Such a feature may be realized through the receiving capability of the HIFU apparatus.

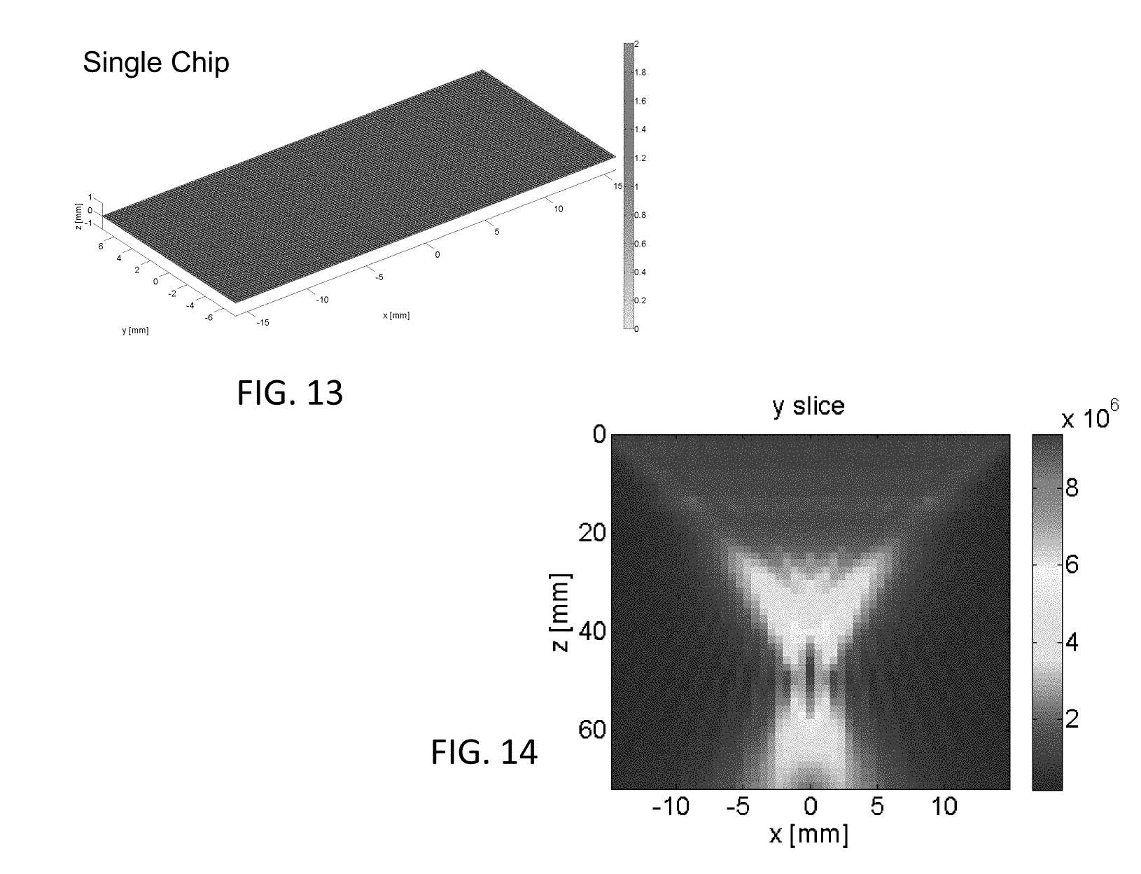

FIGS. 13-18 illustrate various power and acoustic pressure field simulations using different numbers of HIFU transducer chips. From a finite element model (FEM) simulation, transducer surface pressure may currently reach about 1.0 MPa (peak-to-peak) with 100 Vpp (peak-to-peak) pulsing, assuming ideal focusing and discarding medium attenuation. Field simulations were based on physical acoustic wave propagation and beamforming predictions of the pressure and intensity (I_SPPA, spatial-peak, pulse-average) at the focal point.

FIGS. 13 and 14 illustrate pressure and intensity simulations for a single HIFU transducer chip defining a 140×64 array of transducer elements with a 208 μm pitch. The array size is 2.9×1.3 cm². Using a focus depth of 5 cm, the simulated pressure=9.4 MPa (pp), with intensity I_SPPA=0.72 kW/cm².

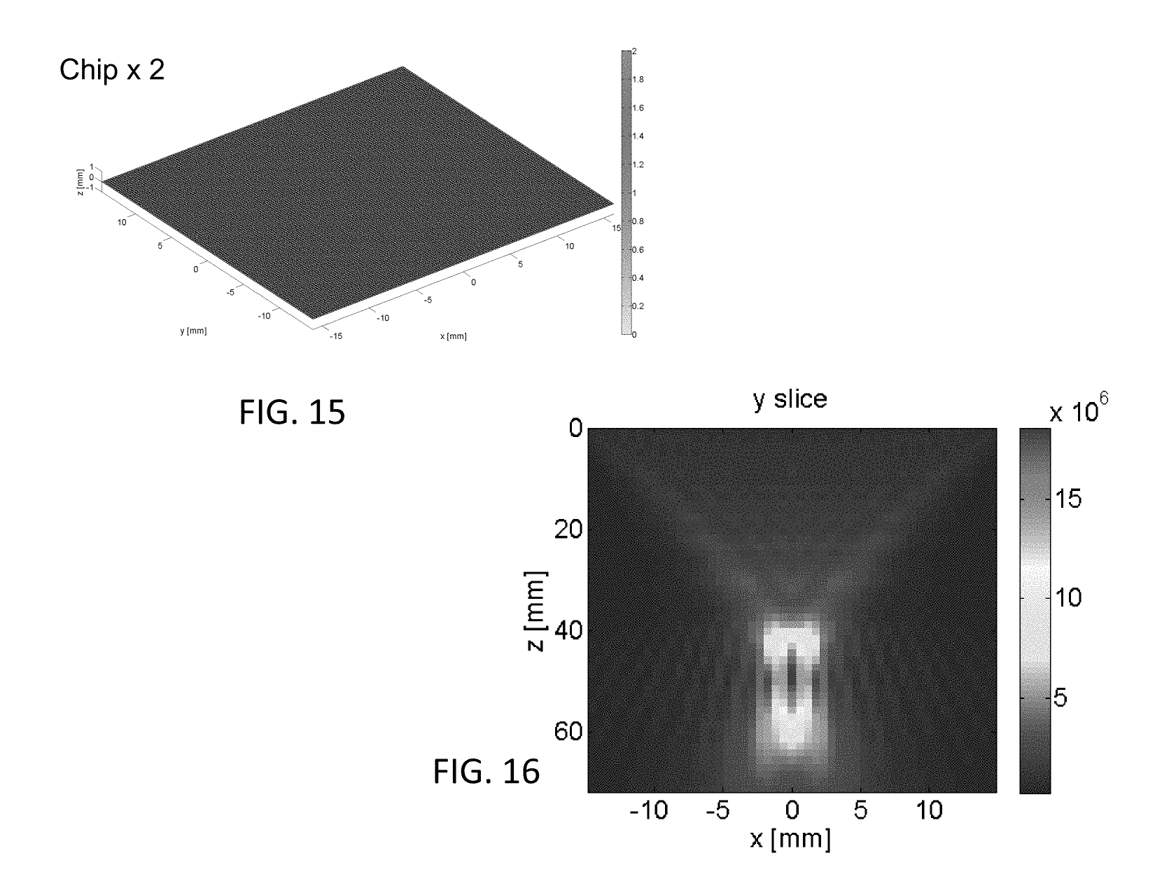

FIGS. 15 and 16 illustrate pressure and intensity simulations for a 2-chip HIFU transducer assembly defining a 140×128 array of transducer elements with a 208 μm pitch. The array size is 2.9×2.7 cm². Using a focus depth of 5 cm, the simulated pressure=18.6 MPa (pp), with intensity I_SPPA=2.80 kW/cm².

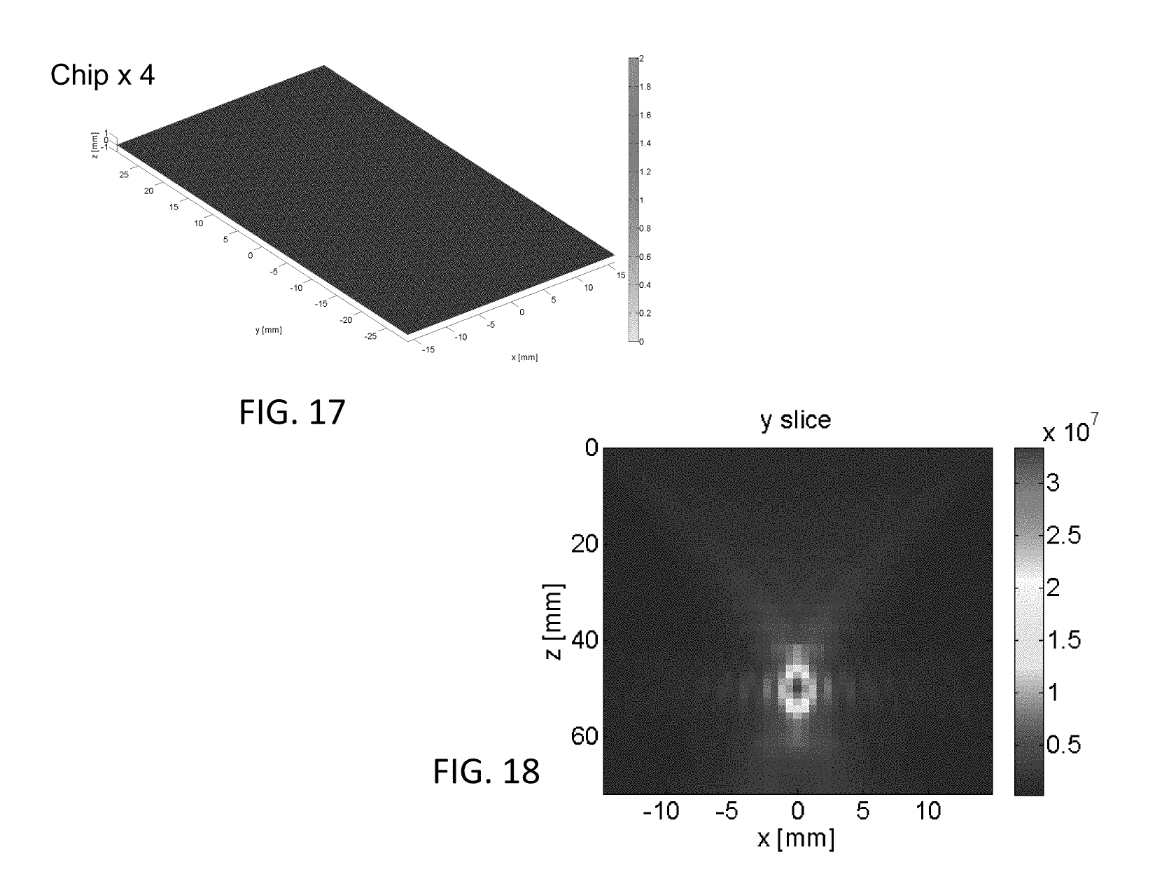

FIGS. 17 and 18 illustrate pressure and intensity simulations for a 4-chip HIFU transducer assembly defining a 140×256 array of transducer elements with a 208 μm pitch. The array size is 2.9×5.3 cm². Using a focus depth of 5 cm, the simulated pressure=33.4 MPa (pp), with intensity I_SPPA=9.07 kW/cm².

One exemplary embodiment of a HIFU apparatus may provide advantageous pressures by employing at least 4 full-reticle chips tiled together with coherent delays between the chips. Non-limiting examples of tiling ultrasound chips are described in U.S. Pat. No. 9,351,706, which is assigned to the Assignee of the present application and is incorporated herein by reference in its entirety. From a power perspective, a 100-200V multi-level, charge recycling pulser may be used. In one specific example, a 5-level pulser design fits a 400 μm×400 μm element size. A peak power consumption estimate per chip corresponds to 2 pF*140*64*(100V)²*2 MHz/4=90 W.

The techniques described herein are exemplary, and should not be construed as implying any particular limitation on the present disclosure. It should be understood that various alternatives, combinations and modifications could be devised by those skilled in the art from the present disclosure. For example, steps associated with the processes described herein can be performed in any order, unless otherwise specified or dictated by the steps themselves.

What is claimed is:

1. An ultrasound imaging system, comprising:
a first ultrasonic transducer assembly configured to deliver high intensity focused ultrasonic (HIFU) energy to a point of interest within a subject; and
a second ultrasonic transducer assembly configured to perform imaging of the subject.

2. The system of claim 1, further comprising a circuit board to which the first ultrasonic transducer assembly is mounted.

3. The system of claim 2, wherein the second ultrasonic transducer assembly is coupled to the circuit board.

4. The system of claim 3, wherein the first ultrasonic transducer assembly comprises a plurality of HIFU transducer chips.

5. The system of claim 4, wherein the plurality of HIFU transducer chips is arranged on the circuit board so as to surround the second ultrasonic transducer assembly.

6. The system of claim 1, further comprising a cooling block in thermal contact with the first ultrasonic transducer assembly.

7. A system, comprising:
a plurality of high intensity focused ultrasonic (HIFU) units, each configured to deliver high energy focused ultrasound energy to a point of focus; and
receive circuitry configured to determine a relative alignment between individual HIFU units so as to implement a self-calibration with respect to a transmit phase of the individual HIFU units.

8. The system of claim 7, wherein the plurality of HIFU units are arranged such that the point of focus is within a brain of a subject.

9. The system of claim 7, wherein the plurality of HIFU units are arranged in a non-coplanar fashion.

10. The system of claim 7, wherein the plurality of HIFU units are arranged to accommodate a rounded anatomical structure.

11. An ultrasound imaging system, comprising:
an ultrasound probe having an acoustic lens; and
a housing configured to receive at least the acoustic lens of an ultrasound probe.

12. The ultrasound imaging system of claim 11, wherein the housing further comprises an aperture configured to receive at least a portion of the ultrasound probe.

13. The ultrasound imaging system of claim 12, wherein the housing is further configured to receive the acoustic lens to thereby form a co-planar surface of a surface of the housing and the acoustic lens.

14. The ultrasound imaging system of claim 11, wherein the housing further comprises at least one cooling line to deliver a coolant to the housing.

15. The ultrasound imaging system of claim 11, wherein the housing further comprises a connector to deliver power to at least one of the housing and the ultrasound probe.

16. The ultrasound imaging system of claim 11, wherein the housing further comprises a transducer chip array.

17. The ultrasound imaging system of claim 16, wherein the transducer chip array comprises a plurality of capacitive micromachined ultrasonic transducers (CMUTs) formed from a semiconductor substrate.
